(19) United States
(12) Patent Application Publication
(10) Pub. No.: US 2011/0300606 A1
(43) Pub. Date: Dec. 8, 2011

(54) FORMULA OF MEDIUM FOR CULTURING CORDYCEPS SPP.

(76) Inventors: Chih-Hui YANG, Chiayi City (TW); Keng-Shiang Huang, Chiayi County (TW); Ya-Hui Hsieh, Changhua County (TW)

(21) Appl. No.: 12/794,457

(22) Filed: Jun. 4, 2010

(51) Int. Cl. C12N 1/14

(52) U.S. Cl. 435/256.8

(57) ABSTRACT

A formula of medium for culturing Cordyceps spp. comprises 1˜6 wt % of carbon sources, 0.8˜51.5 wt % of nitrogen sources, 2˜12 wt % of alginate and 86.2˜95.2 wt % of solvent.

### BACKGROUND OF THE INVENTION

1. Field of the Invention

The present invention relates to a formula of medium, particularly to a formula of medium for culturing Cordyceps spp.

2. Description of the Related Art

Cordyceps spp. is a genus of ascomycete fungi distributed worldwide, included more than 400 described species. Cordyceps fungi are all parasitic, mainly on insects, plants, fungi and some arachinidas. According to the records, few Cordyceps fungi are considered a herbal or traditional Chinese medicine which have interesting pharmacological properties and been commonly used in oriental countries like China, Korea and Japan for long history. Among them, the most popular, also the best therapeutic species is Cordyceps sinensis, also known as caterpillar fungus.

Generally, caterpillar fungus is resulted from a parasitic relationship between a Cordyceps fungous and its host, usually a larva of insects. When the Cordyceps fungous infect its host, the mycelium of the Cordyceps fungous may invade the host tissue and finally replace the body of the host to form a complex-shape. For example, a Cordyceps sinensis, named dong chong xia cao in Chinese, generally known by public is derived from a fungous of Cordyceps sinensis germinating in a living larva of Hepialus armoricanus, such as Oberthur, presenting in a worm-shape in the winter but turning to plant-shape in the summer.

In recent decades, the biochemical and pharmaceutical interests of Cordyceps fungi have been concerned, especially for its therapeutic effects on circulatory system, immunity, cardiovascular system and endocrine system. In western countries, it is believed that Cordyceps fungi are a kind of medicinal mushroom with many prized pharmacological substances which are sufficient to treat for immunodeficiency or cancer. Although it is still unclear about the therapeutic mechanism of Cordyceps fungus involved in, most scientists suggest that some active substances produced by Cordyceps spp., such as cordycepin and adenosine, tends to be a biochemical indicator of medical values in Cordyceps fungi. Furthermore, it is also proved that polysaccharide of Cordyceps fungi extracted from fermentative medium shows significant therapeutic effects on immune defects.

However, due to particular requirement of environment for natural-grown Cordyceps fungi, the production of natural-grown Cordyceps fungi is quite deficient. Moreover, out of excessive exploitation and climate change of the medicinal mushroom in these days, the natural-grown Cordyceps fungi become extremely rare and costly. Hence, some artificial cultivation methods of Cordyceps fungi have developed rapidly in order to cultivate on an industrial scale of Cordyceps fungi for its medicinal utility. In conventional technique, the artificial cultivation methods for Cordyceps fungi are divided into two types including an in vivo type and an in vitro type of cultivation. For in vivo cultivation, Cordyceps fungi are directly inoculated and cultivated in larvae of host insects. However, due to the difficulty of insect inoculation and industrial manufacture of in vivo cultivation, it has rarely been used in the biomedical industry currently. On the other hand, for in vitro cultivation, some medium may be used to replace the host body for the development of Cordyceps fungi. Generally, according to the different species of cultivation Cordyceps fungi, diverse medium, such as YMM (YM medium), MEM (maltose extraction medium) and PEM (potato extraction medium) are developed and prepared followed by different formula of various carbon sources, nitrogen sources and some other nutrition likes vitamin (B1 or B6 for example), organism (phosphate for example) or inorganic substances (calcium sulfate for example). The carbon sources usually used in the medium can be dextrose, maltose, lactose, monosaccharide, disaccharide, dextrin and starch. The nitrogen sources used in the medium can be yeast powder, soy bean powder, peptone and wheat bran. Also, three phase of medium are generally prepared and used in accord with various requests of cultivation products, including liquid phase, solid phase and semi-solid phase of medium as following described.

The liquid medium for culturing Cordyceps fungi is preferable for fungi fermentation. It is capable to obtain plenty amount of fermentation products under low cost, short culturing period and easy process. However, with the use of the liquid medium, it has problem to obtain industrialized scale of fermentation production, also for some particular species of Cordyceps fungi the fungi viability may be poor. For example, as defined in Taiwan patent no. 200412371 discloses a method and a media for liquid culturing the mycelial of Cordyceps sinensis, wherein a primary substantial of the media is an agricultural product comprising polysaccharide. However, the media is not only incomplete at improving the fermentation rate of the culturing Cordyceps sinensis, but necessitate an additional process converting the polysaccharide of the agricultural product into monosaccharide for Cordyceps sinensi to use.

The solid medium is prepared from adding a colloidal material into a liquid medium, which can provide an adhesion for culturing Cordyceps fungi to enhance the development of fungous carpophore. However, compare to the liquid medium, the manufacturing process and culturing condition of the solid medium are not easy to control, which may result in time-consuming and contamination in some situation. For example, as defined in Taiwan patent no. I231825 disclose a solid state fermentation (SSF) method for fungal cultivation, such as Cordyceps sinensis and Antrodia camphorata, wherein three different media are used in the SSF method including a solid media, a liquid media and a solid fermentation media. Generally, a fungus is sequentially inoculated in the solid media, transferred in the liquid media and the solid fermentation media for further assigning a preferable culturing system for fungal fermentation. However, even going through the complicated process of the SSF method, it is still less efficient in preventing the contamination problem and promoting the production and activity of fermented products of culturing fungi.

The semi-solid medium contains both liquid medium and solid substrate of nutrition, such as rice and wheat, wherein the solid substrate of nutrition can provides an adhesion, also a nutrition source for the culturing Cordyceps fungi. As defined in Taiwan patent no. I242044 discloses a formula of liquid medium comprising a solid nutrition which can provide substantial nutrition also, an adhesion for culturing fungous. The formula of liquid medium is tended to provide a semi-solid liquid medium both contained solid state and liquid medium which can share the advantages as in solid medium and liquid medium. However, for the most semi-solid liquid medium, they still have the disadvantages of the solid medium and liquid medium, therefore, inadequate to provide a most preferable cultivation environment for Cordyceps fungi.

As a result, although diversity of the conventional medium have been developed and applied on the industry for industrial culturing and producing of medicinal products of Cordyceps fungi. Nevertheless, most medium are inconvenient in process and still poor in promoting the fermentation efficiency and mycelium viability of culturing Cordyceps fungi. Hence, there is a need of improving the conventional medium for culturing Cordyceps fungi to advance the development of artificial cultivation methods of Cordyceps fungi.

### SUMMARY OF THE INVENTION

The primary objective of this invention is to provide a formula of medium for culturing Cordyceps spp., which can improve the viability of Cordyceps spp. under artificial cultivation environment.

The secondary objective of this invention is to provide a formula of medium for culturing Cordyceps spp., which can increase the production of fermented production under artificial cultivation environment.

Another objective of this invention is to provide a formula of medium for culturing Cordyceps spp., which can increase the production of polysaccharides of Cordyceps spp. under artificial cultivation environment.

Another objective of this invention is to provide a formula of medium for culturing Cordyceps spp., which can increase the production of mycelium of Cordyceps spp. under artificial cultivation environment.

A formula of medium for culturing Cordyceps spp. comprises 1˜6 wt % of carbon sources, 0.85˜1.5 wt % of nitrogen sources, 2˜12 wt % of alginate and 86.2˜95.2 wt % of solvent.

Further scope of the applicability of the present invention will become apparent from the detailed description given hereinafter. However, it should be understood that the detailed description and specific examples, while indicating preferable embodiments of the invention, are given by way of illustration only, since various will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a bar chart illustrating the viability of cordyceps spp. in a medium with or without alginate by times;

FIG. 2 is a bar chart illustrating the production of extracellular polysaccharids of cordyceps spp. in a medium with or without alginate by times;

In the various figures of the drawings, the same numerals designate the same or similar parts.

### DETAILED DESCRIPTION OF THE INVENTION

In the present invention, a liquid fermentation medium is prepared according to a formula of medium for culturing Cordyceps spp., which is beneficial to the viability of mycelium, production of polysaccharide and fermented products of Cordyceps spp.. With an inoculum of Cordyceps spp. cultured in the liquid fermentation medium, it is sufficient to maintain the optimum growth conditions and fermentation of the Cordyceps spp..

The formula of medium for culturing Cordyceps spp. in the present invention comprises 1˜6 wt % of carbon sources, 0.85˜1.5 wt % of nitrogen sources, 2˜12 wt % of alginate and 86.2˜95.2 wt % of solvent, wherein the alginate is added in diverse form.

The carbon sources in the formula of medium for culturing Cordyceps spp. in the present invention can be selected from a group of dextrose, sucrose, lactose and maltose, which is high in utility rate mainly for providing a substantial nutrition to Cordyceps spp. as developing. The nitrogen sources in the formula of medium for culturing Cordyceps spp. includes fixed in nitrogen sources, yeast extraction and maltose extraction for example, and changed in nitrogen sources, peptone and corn powder for example, which provides primary nutrition of developing for Cordyceps spp. In additional, the solvent mentioned above can be water, for completely dissolving and mixing up with the carbon sources, nitrogen sources and alginate, wherein the alginate in use can be liquid, solid, powder or any possible form. The detail of the formula of medium for culturing Cordyceps spp. in the present invention is summarized in Table 1.

TABLE 1
the formula of medium for culturing Cordyceps spp.

| Compositions | Ratio (wt %) |
|---|---|
| Dextrose | 1.0~6.0 |
| Malt extract | 0.3~0.5 |
| Peptone | 0.25~0.5 |
| Yeast extract | 0.3~0.5 |
| Alginate | 2~12 |

The alginate used in the present invention is an anionic polysaccharide mainly extracted from seaweed which can absorb and convert water quickly into viscous gel. In this situation, the texture of the liquid fermentation medium in the present invention may become slightly gluey but still in liquid form which provides an adhesion for inoculum of Cordyceps spp. as developing, also a beneficial fermentation environment for metabolism of mycelium. Moreover, for further improving the growth of the inoculum of Cordyceps spp., 0.05 wt % to 0.1 wt % of inorganic substance (such as potassium phosphate and magnesium phosphate) are preferable to add into the liquid fermentation medium in the present invention adjusting to pH 6.

For studying the utility of the liquid fermentation medium on viability of mycelium of Cordyceps spp. in the present invention, two groups of medium are prepared including an alginate group (1), with the formula of medium defined in Table 1; and a control group (2), with a similar formula of medium as defined in Table 1 but without alginate. Meanwhile, inoculums of Cordyceps spp. are differentially inoculated in the medium of the alginate group (1) and the control group (2) culturing at a incubation of 20 to 25° C. and 150 rpm for 14 to 30 days in order to recorder the viability of Cordyceps spp. in two groups.

The inoculums of Cordyceps spp. used in the present invention are Cordyceps militaris, also called northern caterpillar fungus, which is a type species of the genus Cordyceps. The Cordyceps militaris contains similar biochemical component, such as mannitol, adenosine, cordycepin and polysaccharide to Cordyceps sinensis, also with similar medicinal properties like anti-tumor, anti-inflammatory, anti-oxidant activity and immunity regulation to Cordyceps sinensis. Nowadays, fruit bodies of Cordyceps militaris have been mass-produced and well-developed artificially for applying to food industry. However, due to the bottleneck of the conventional artificial technique, it is lack of a vigorous system to maintain the viability of mycelium, also the production of metabolite under an artificial culturing environment.

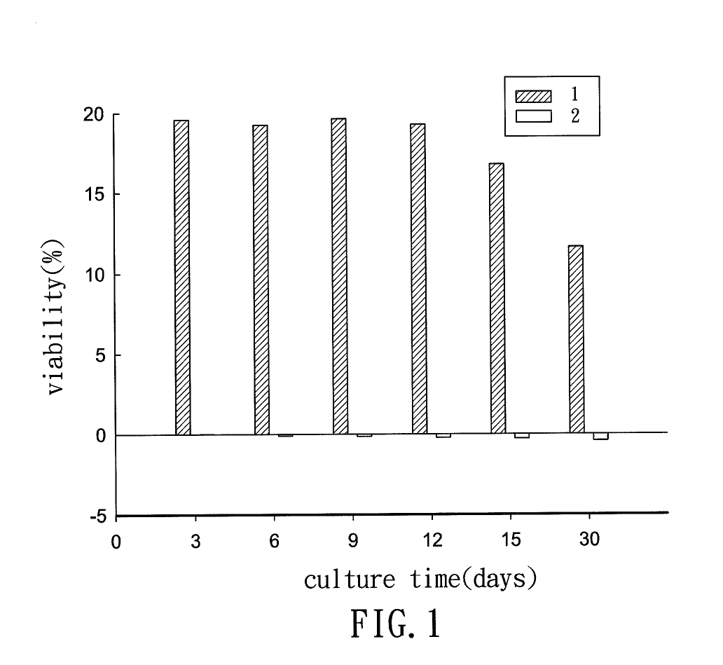

Referring to FIG. 1, the inoculums of Cordyceps spp. cultured in the medium of alginate group (1) shows significant optimum positive growth than that in the medium of control group (2), with +20% of viability in the 3 days post inoculation, also keeping at >10% of viability lasting for the rest of culturing period. On the other hand, the viability of Cordyceps spp. cultured in the medium of control group (2) is getting worse by day during the culturing period, which shows negative growth from the 6 days post inoculation. It is suggested that adding alginate into medium is beneficial to the viability of Cordyceps spp. which can turn the negative growth of Cordyceps spp. into positive development. Therefore, it is believed that the formula of medium for culturing Cordyceps spp. in the present invention is sufficient to improving the quality of development of Cordyceps fungi under artificial culturing environment.

Additionally, for further studying the effects of the liquid fermentation medium on production and activity of mycelium of Cordyceps spp. in the present invention, the inoculums of Cordyceps spp. are inoculated in two groups of medium [including the alginate group (1) and control group (2) as described above] again for regularly analyzing the culturing medium of two groups. In the present invention the culturing medium from two groups are collected and filtered for periodical monitoring and recording the production of polysaccharide in the culturing medium by a phenol-sulfuric acid method.

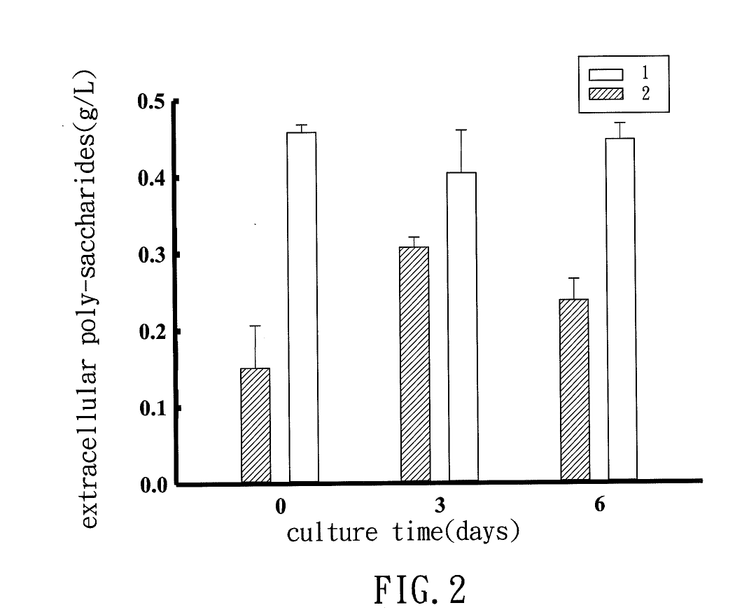

Referring to the FIG. 2, the inoculums of Cordyceps spp. cultured in the medium of the alginate group (1) reveals higher production of polysaccharide than that produced in group (2) during post inoculation period, with around 0.4 to 0.45 gram of polysaccharide produced in per liter of culturing medium. Also, the polysaccharide produced in group (1) has been tested to perform well in activity. On the other hand, the inoculums of Cordyceps spp. cultured in the control group (2) only produces no more than 0.3 grams of polysaccharide in per liter of culturing medium from 0 to 6 days post inoculation, also with low in activity. It is suggested that adding alginate into the liquid fermentation medium is much advantageous to the production and activity of polysaccharide produced by Cordyceps spp. which presents dramatic increases of production of polysaccharide than that in the medium without alginate. Hence, it is believed that the formula of medium for culturing Cordyceps spp. in the present invention truly can improve the production of metabolic in Cordyceps fungi under an artificial culturing system.

In summary, the liquid fermentation medium prepared from the formula of medium for culturing Cordyceps spp. in the present invention can positively promote the viability of mycelium, the production of metabolic and activity of fermented products of Cordyceps fungi. Accordingly, it is sufficient to obtain high production and better activity of metabolic products of Cordyceps fungi via the liquid fermentation medium, which is beneficial to develop Cordyceps fungi related products or researches on anti-carcinoma, anti-oxidation and anti-age for biomedical utility.

Through the present invention, the formula of medium for culturing Cordyceps spp. contains carbon sources, fixed in nitrogen sources, changed in nitrogen source as involved in general formula of medium and an alginate, wherein the alginate can absorb and convert water into gelable texture to provide a adhesion of growth, enough nutrition and preferable environment of fermentation for the culturing Cordyceps spp. Therefore, with the formula of medium for culturing Cordyceps spp., it is efficient to control the production of mycelium and polysaccharide of Cordyceps spp. which can be more economical and convenient cultivated on an industrial scale for its medicinal value.

Although the invention has been described in detail with reference to its presently preferred embodiment, it will be understood by one of ordinary skill in the art that various modifications can be made without departing from the spirit and the scope of the invention, as set forth in the appended claims.

What is claimed is:

1. A formula of medium for culturing Cordyceps spp. comprising:
1˜6 wt % of carbon sources;
85˜1.5 wt % of nitrogen sources;
2˜12 wt % of alginate; and
86.2˜95.2 wt % of solvent.

2. The formula of medium for culturing Cordyceps spp. as defined in claim 1, wherein the carbon sources in the formula is dextrose.

3. The formula of medium for culturing Cordyceps spp. as defined in claim 1, wherein the nitrogen sources in the formula is selected from a group of malt extract, yeast extract and peptone.

4. The formula of medium for culturing Cordyceps spp. as defined in claim 1, wherein the formula of medium for culturing Cordyceps spp. further comprises 0.05˜0.1 wt % of potassium phosphate or magnesium phosphate and adjust medium to pH 6.

5. The formula of medium for culturing Cordyceps spp. as defined in claim 1, wherein the solvent in the formula is water.
